(19) United States
(12) Patent Application Publication
(10) Pub. No.: US 2009/0005651 A1
(43) Pub. Date: Jan. 1, 2009

(54) Portable systems, devices and methods for displaying varied information depending on usage circumstances

(75) Inventors: Suzanne Marie Ward, Portland, OR (US); Richard Alan Davis, Chesterfield, MO (US); Peter Michael Galen, Portland, OR (US)

Correspondence Address: Hiscock & Barclay, LLP, One Park Place, 300 South State Street, Syracuse, NY, 13202-2078, US

(73) Assignee: Welch Allyn, Inc., Skaneateles Falls, NY (US)

(21) Appl. No.: 11/823,177

(22) Filed: Jun. 27, 2007

(51) Int. Cl. A61B 5/00

(52) U.S. Cl. 600/300

(57) ABSTRACT

A portable monitor device includes an integral display to act portably to display various information, the portable monitor device being configured for placement selectively in a docking station. In the docking station, the monitor device automatically provides information relative to at least one larger, external display device, and the display of the monitor device can be used as a user interface in conjunction with the operation of various display and system related functions.

### FIELD OF THE INVENTION

This invention relates to display devices and systems incorporating the same, and, more particularly, to monitoring systems incorporating portable display devices that display varied information depending on usage circumstances. Still more particularly, the invention relates to medical monitoring systems incorporating display devices that display certain medical information depending on whether or not the display device is being used portably.

### BACKGROUND OF THE INVENTION

In recent decades, numerous devices such as cellular telephones, music players, PDAs and even computers have become increasingly portable, whereby they can be operated—at least for certain durations—using battery power or other untethered power sources. A primary goal in rendering these devices portable is to provide people with the ability to enjoy them in a manner with which they are familiar, but with fewer limitations as to the locations in which the devices must be utilized. In general, such portable devices provide the same or similar features and usage options whether they are being used portably or not.

Increasingly, more devices have become portable as technology has advanced and people have become accustomed to seeing and using portable devices and more accepting of their reliability. Among such newer portable devices are those that are utilized in medical settings. For example, certain patents (see, e.g., U.S. Pat. Nos. 6,591,135 to Palmer et al. and 6,829,501 to Nielsen et al. and U.S. Patent Application Publication Nos. 2002004/0147818 by Levy et al., 2005/0033124 by Kelly et al., and 2005/0051168 by DeVries et al.) disclose portable medical equipment (e.g., a monitor, a ventilator) that can be placed in a docking station when not being used portably in order to switch from a wireless to a wired connection, to connect the devices to a communication network or other network devices, and/or to recharge the untethered power source of the portable equipment.

Like the other aforementioned portable medical equipment, however, portable medical monitors display identical information whether being used portably or not. This is true with regard to the monitors described in the above-noted patents and patent publications, as well as currently manufactured monitors such as the Pick and Go® patient monitoring systems that are commercially available from Draeger Medical, Inc. of Tellford, Pa. USA.

The fact that portable medical equipment displays the same information regardless of whether or not it is being used portably can be suboptimal for several reasons. Most notably, although the information displayed by portable medical equipment generally is useful in a portable setting, such information, by itself, may be incomplete, superfluous or otherwise unsatisfactory if the portable equipment is used in a non-portable setting. This is not ideal for hospitals and other facilities, which often have stretched budgets and would prefer equipment that can be used in a variety of settings and under a variety of conditions.

Thus, there is a need for portable display devices, including portable medical equipment, wherein the information or content that is produced by and/or displayed on such portable equipment can vary (e.g., automatically) depending on usage conditions/settings.

### SUMMARY OF THE INVENTION

These and other needs are met by a monitoring system, which, in accordance with an exemplary aspect, comprises: (a) a first display device (e.g., a computer monitor) (b) a second display device (e.g., a medical patient monitoring device), and (c) a docking station that is adapted to receive the second display device. The second display device is operable in one of a docked mode (i.e., while the second display device is docked within the docking station) and an undocked mode (i.e., while the second display device is undocked from the docking station), such that while in the undocked mode the second display device operates as a primary display device for the system, and such that while in the docked mode the second display device operates as a user interface or a secondary display (e.g., a touch screen) and the first display device operates as the primary display device for the system.

In accordance with this, and, if desired, other exemplary systems, the second display device can include at least one power source (e.g., at least one battery) for powering the second display device while the second display device is in the undocked mode. Additionally or alternatively, the second display device can further comprise: (a) a plurality of modules, each of which is adapted to receive at least one signal, and (b) a microprocessor that includes software to permit the at least one signal to be displayed on the second display device in at least one display format. Also additionally or alternatively, the system can further comprise a control unit that is adapted to sense when the second display device is in the undocked mode, wherein the control unit includes a communicating means for communicating display data to the first display device while the second display device is in the docked mode.

Also in accordance with this, and, if desired, other exemplary systems, the user interface of the second display device can include a plurality of user actuable controls, at least some of which enable a user to maneuver within and select from information displayed on the second display device. By way of example, one or more of the plurality of actuable controls can be buttons, such as one or more of a power button, a mute button, an image save button, and a perform procedure button.

In further accordance with this, and, if desired, other exemplary systems, the docking station can include a receiving area to receive the second display device, wherein the receiving area can include at least one detent. Additionally or alternatively, the docking station can include a receiving slot and/or a dispensing slot.

These are other needs also are met by a medical monitoring system, which, according to an exemplary aspect, comprises: (a) a docking station that has a receiving area, and (b) a monitor device that is adapted to fit within the receiving area of the docking station, wherein the monitor device includes a display screen on which medical information is displayed, and also wherein the medical information is automatically modified upon removal of the monitor device from the docking station.

In accordance with this exemplary medical monitoring system, and, if desired, other exemplary systems, the monitor device can include a plurality of receiving areas, each of which enables connection between the monitor device and equipment adapted to measure at least one physiological parameter. Such physiological parameters can include, by way of non-limiting example, pulse oximetry, end-tidal CO₂, heart rate, non-invasive blood pressure, invasive blood pressure, and body temperature.

These and other needs are still further met by another exemplary medical monitoring system, which, according to an exemplary aspect, comprises: (a) a docking station that has a receiving area; and (b) a monitor device that is adapted to fit within the receiving area of the docking station, wherein the monitor device comprises: (1) a display screen, (2) a plurality of receiving areas, each of which enables connection between the monitor device and equipment that is adapted to measure at least one physiological parameter, (3) a plurality of modules, each of which is adapted to receive at least one signal representative of medical information, (4) a microprocessor that includes software to permit each of the at least one signal to be displayed on the monitor device in at least one display format, and (5) a user interface that includes a plurality of user actuable controls. In accordance with this system, the medical information is automatically modified upon removal of the monitor device from the docking station.

Still other aspects, details, embodiments and advantages of these various exemplary aspects are discussed in detail below. Moreover, it is to be understood that both the foregoing general description and the following detailed description are merely illustrative examples of various embodiments, and are intended to provide an overview or framework for understanding the nature and character of the claimed embodiments. The accompanying drawings are included to provide a further understanding of the various embodiments, and are incorporated in and constitute a part of this specification. The drawings, together with the description, serve to explain the principles and operations of the described and claimed embodiments.

### BRIEF DESCRIPTION OF THE DRAWINGS

For a fuller understanding of the nature and desired objects of the present invention, reference is made to the following detailed description taken in conjunction with the accompanying figures, wherein like reference characters denote corresponding parts throughout the views, and in which:

FIG. 1 is an isometric view of an exemplary external patient monitoring system that includes an external display and a monitor device that is docked within a docking station;

FIG. 2 is a front view of the external display of FIG. 1 while the external display is displaying exemplary primary medical information;

FIG. 3 is an isometric view of the docking station of FIG. 1;

FIG. 4 is an enlarged front view of the monitor device of FIG. 1;

FIGS. 4A-4F are front views of the display screen of the monitor device of FIG. 1; while it is displaying secondary medical information

FIG. 5 is a front view of the display screen of the monitor device while it is displaying primary medical information;

FIG. 6 is a schematic drawing showing an exemplary block diagram of the monitor device of FIG. 1; and

FIG. 7 is a block diagram of an exemplary architecture of the docking station of FIG. 3.

### DETAILED DESCRIPTION

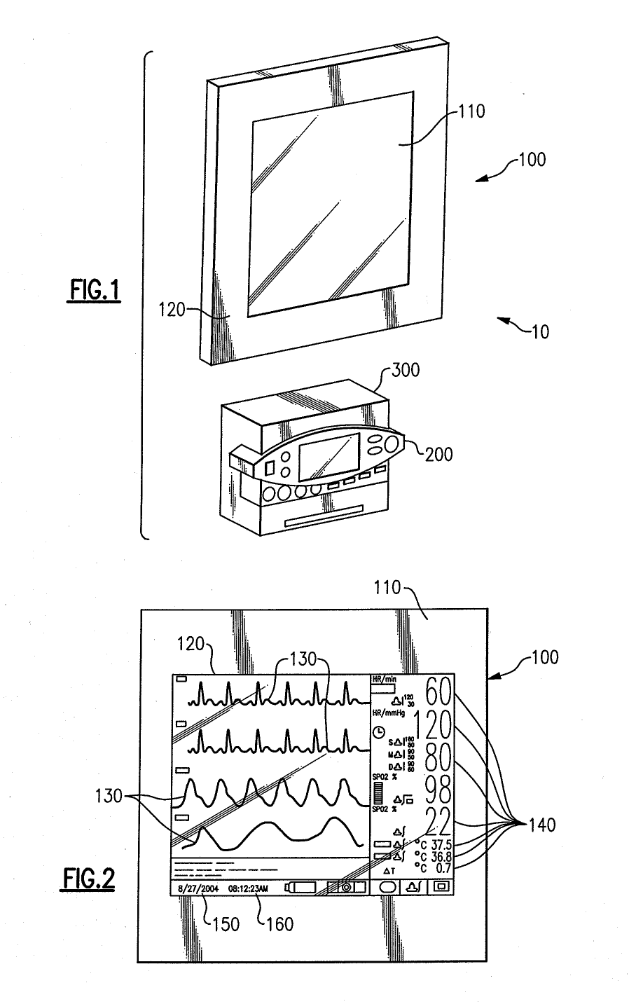

Referring initially to FIG. 1, an exemplary embodiment of a monitoring system 10 includes an external display 100 and a monitor device 200. The monitor device 200 can be used portably or while docked within a docking station 300, each as discussed below. By way of non-limiting example, the monitoring system 10 can be utilized to convey medical information, such as diagnostic or assessment information regarding one or more patients in a hospital or other medical diagnosis or treatment facility. It is understood, however, that the FIG. 1 system 10 can have other arrangements, and can be used for other purposes and/or in other medical-based settings without undue experimentation. It is further understood that the system 10 can include other components (e.g., additional monitors, medical equipment or devices, and the like) in addition to the external display 100, the monitor device 200 and the docking station 300 depicted in FIG. 1. Alternatively, the external display 100 can be omitted from the system 10, if desired.

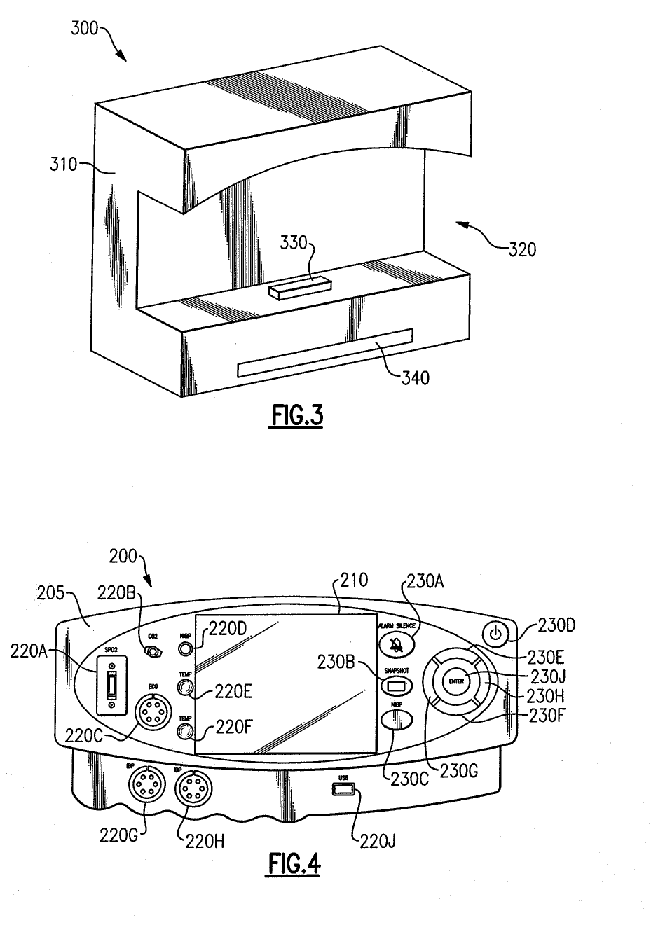

In accordance with the exemplary system 10 shown in FIGS. 1 and 2, the external display 100 includes a display screen 110 for displaying certain medical information and/or data, and an optional border 120 surrounding the screen. The dimensions of the external display 100, its screen 110 and, if included, its border 120 can vary due to several factors, including, but not limited to, design preference and actual or intended usage environment. Generally, the external display 100 is a non-portable device, whereby it is intended to be used or is limited to being used while connected to or otherwise in communication (e.g., by one or more wires or cords) with a tethered power source; however, if instead desired, the external display can be wirelessly powered as is known in the art.

The external display 100 also is connected to or is in communication with one or more pieces or equipment, gauges, modules and/or communication networks as is generally known in the art (see, e.g., U.S. Pat. Nos. 6,988,989, 6,616,606, 6,544,174, and 6,544,173, each of which is incorporated by reference in its entirety herein) and so as to enable the external display to visually convey medical information and/or data that is gathered by or from medical testing, reading or measurement equipment. Such connection(s) can occur as is generally known in the art, e.g., by wires or cords that connect either to the external display through one or more inputs (not shown) of the external display 100, or that connect to a device (e.g., a computer) to which the external display is connected. Generally, but not necessarily, the screen 110 of the external display 100 continually or periodically displays some or all of the medical information or data in real-time.

The medical information and/or data displayed on the screen 110 of the external display 100 is referred to herein as “primary medical information” and can include, by way of non-limiting example, certain information pertaining to the immediate or ongoing health and/or prognosis of a patient, whereby such information is periodically or continually monitored and assessed by medical personnel and/or equipment. As depicted in FIG. 2, such primary medical information generally includes, but need not be limited to, one or more patient “vital signs,” which can be presented as waveforms 130 and/or as numerics 140. The specific choice of which “vital signs” is/are shown on the screen 110 can vary depending on several factors such as the actual or suspected diagnosis or prognosis of the patient; however, exemplary “vital signs” data can include, but is not limited to, any, some or all of a pulse oximetry (SPO₂) reading, an electrocardiogram (ECG) reading, an end tidal CO₂ reading, a heart rate reading, one or more invasive and/or non-invasive blood pressure measurements, and a body temperature measurement.

Also as shown in the exemplary embodiment of FIG. 2, the screen 110 of the external display 100 can visually depict additional information in addition to primary medical information, wherein such additional information can be—but need not be—medical in nature and can include, by way of non-limiting example, the current date 150, the current time 160, and/or patient demographics (e.g., name, age, race, marital status, date of birth, room number, primary care physician, treating physician(s), and/or other patient data that is deemed relevant on a general or case-by-case basis).

Whereas the external display 100 generally is non-portable, the monitor device 200, as described herein, generally can be used both portably, or while docked within a docking station 300 (i.e., in a “docked” mode/condition as shown in FIG. 1). The terms “undocked” and “dedocked” are used interchangeably herein to refer to the monitor device 200 while it is being used portably. While in a docked condition, the monitor device 200 can be placed in proximity of (e.g., within visual range of) the external display 100, at a more remote visual location away from the external display, or can be used without an external display.

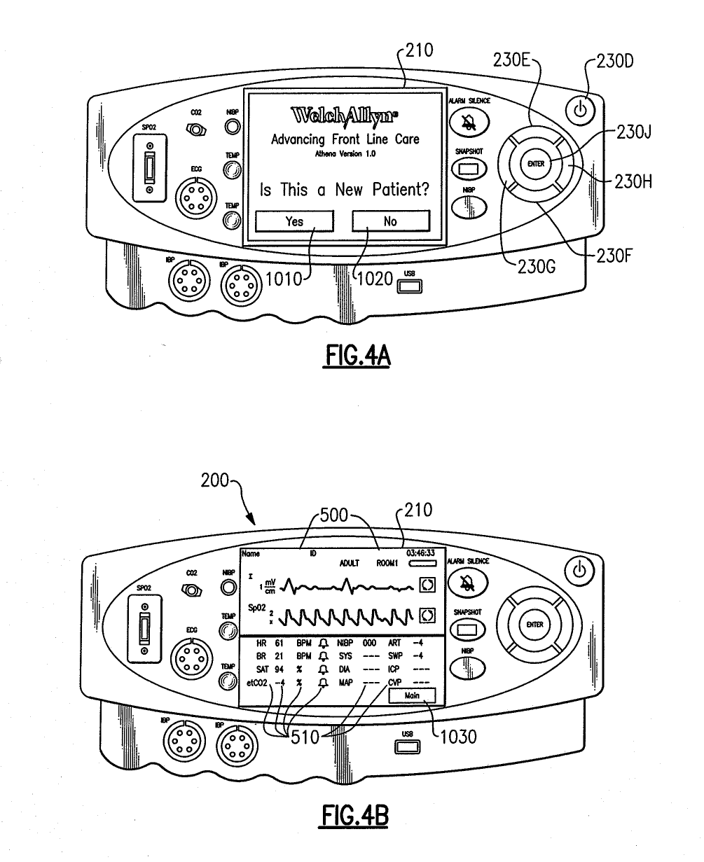

An exemplary docking station 300 for the monitor device 200 is shown in detail in FIG. 3. The docking station 300 includes a main body 310 and a receiving area 320, wherein the receiving area is shaped to physically accept and accommodate the monitor device 200. In accordance with the exemplary embodiment of FIG. 3, at least one detent mechanism 330 or other suitable locking element is included within the receiving area 310 so as to inhibit the monitor device 200, once docked, from inadvertently becoming dislodged and/or disconnected from the docking station 300, yet also to allow the monitor device to be selectively removed (i.e., undocked/dedocked) from the docking station as desired (e.g. to function portably). Generally, the docking station 300 is powered by being connected to or otherwise in communication (e.g., by one or more wires or cords) with a tethered power source; however, if instead desired, the docking station can be wirelessly powered as is known in the art.

The docking station 300 can have other optional features, including, but not limited to, a slot 340 (see FIG. 3), which, depending on the specific design of the docking station, can be adapted to dispense information (e.g., printed paper) relating to the patient or can be configured to receive or accept a device (e.g., a hard disk, floppy disk, jump drive or other information storage medium so as to allow data to be loaded onto or downloaded from the docking station). It should be noted that the docking station 300 can have a different physical configuration from that which is shown in FIG. 3 (e.g., as a matter of design choice or to enable docking of a monitor device 200 having a different design) without undue experimentation.

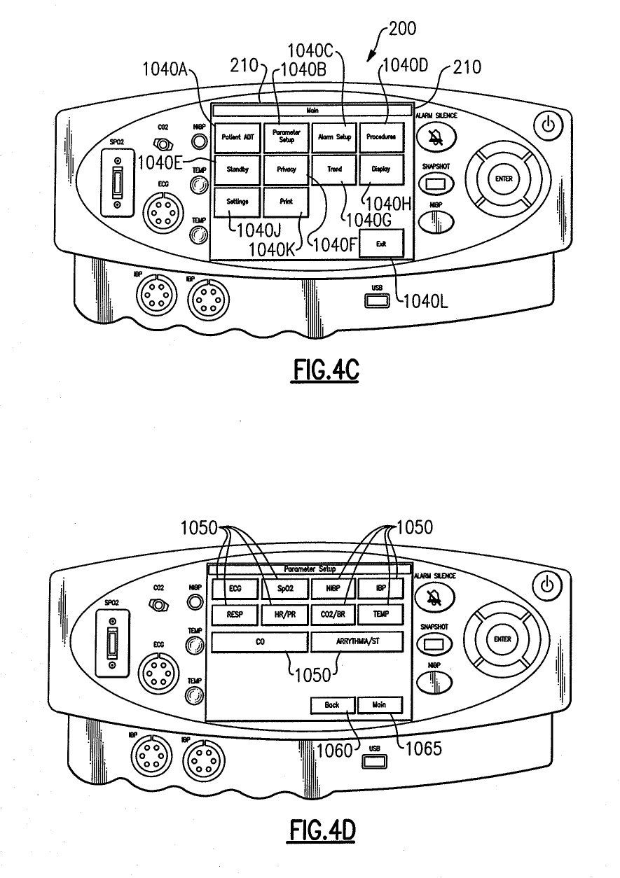

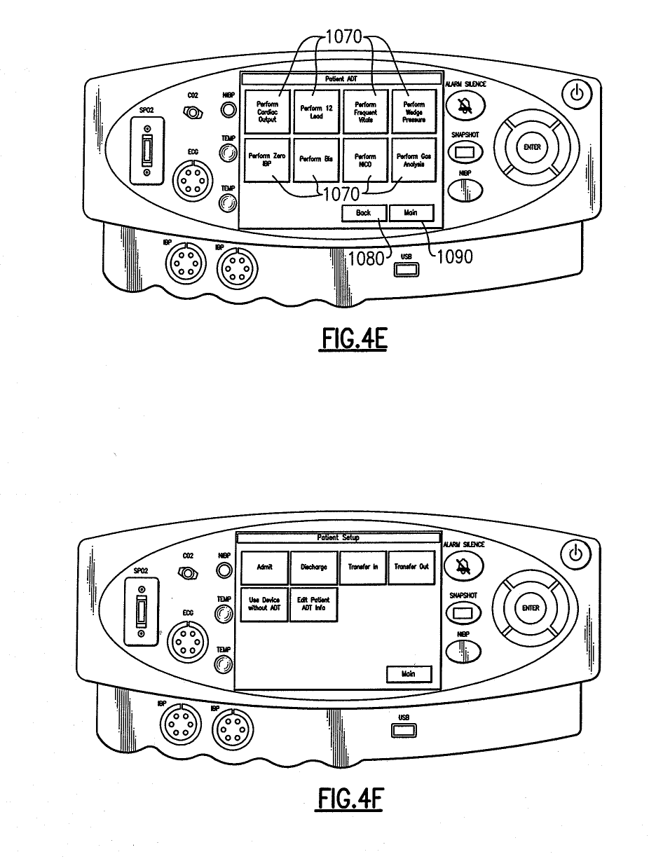

Referring now to FIG. 4, the front panel 205 of the monitor device 200 of FIG. 1 is shown. The front panel 205 includes a display screen 210, one or more connection/receiving areas (e.g., ports) 220 shaped to enable connection (e.g., by a wire or cord) to one or more pieces of medical equipment, and a user interface. The user interface can include, e.g., one or more buttons 230 or other user input/actuation elements or controls, which, when pressed or otherwise actuated by a user, cause one or more predetermined actions to occur, as explained below.

In the exemplary embodiment of FIG. 4, the monitor device 200 includes a plurality of receiving areas 220, such as ports, namely: at least one receiving port 220A to enable connection to pulse oximetry measurement/reading equipment (not shown), at least one receiving port 220B to enable connection to end-tidal CO₂ measurement/reading equipment (not shown), at least one receiving port 220C to enable connection to electrocardiogram measurement/reading equipment (not shown), at least one receiving port 220D to enable connection to non-invasive blood pressure measurement/reading equipment (not shown); two or more receiving ports 220E, 220F to enable connection to different core body temperature measurement/reading equipment (e.g., one piece of equipment which measures/reads core body temperature at the head and another piece of equipment which measures/reads core body temperature at an extremity) (neither shown), two or more receiving ports 220G, 220H to enable connection to different invasive and/or non-invasive blood pressure measurement/reading equipment (not shown), and one or more additional optional receiving ports 220J to enable connection to one or more additional devices (e.g., a printer, a ventilator, etc.) (not shown). Any, some or all of the various receiving ports 220 of the monitor device 200 can be labeled textually, such as is shown in FIG. 4, and/or pictorially.

The exemplary monitor device 200 of FIG. 4 includes, as part of its user interface, a plurality of user actuable controls 230 (e.g., buttons). In this exemplary embodiment, the user actuable controls 230 are buttons, namely: a first button 230A, which, when pressed, mutes a sounding alarm (which sounds, e.g., when a patient reading exceeds a prestored threshold), a second button 230B, which, when pressed, saves into memory an image currently being displayed on the screen 210, a third button 230C, which, when pressed, causes a medical procedure (e.g., a non-invasive blood pressure measurement) to be performed on demand, and a fourth button 230D, which, when pressed, causes the monitor device to power up or down. The user interface portion of the monitor device 200 also can still further include one or more directional buttons, such as an up button 230E, a down button 240F, a left button 240G and/or a right button 240H, each of which, when pressed, causes a cursor or other selection icon displayed on the screen 210 of the monitor device to move in a corresponding direction. Moreover, the monitor device 200 can yet still further include an enter button 230J, which, when pressed, enables a user to select an option currently highlighted or displayed on the screen 210 of the monitor device. Any, some or all of the various buttons 230 can be labeled textually, such as is shown in FIG. 4, and/or pictorially. Moreover, any or all of the buttons 230 can be replaced with other types of user actuable controls, such as knobs and/or levers.

Referring again to FIG. 1, in this exemplary configuration of the system 10 the monitor device 200 is in a docked mode and serves one or more functions or purposes that differ from or are in addition to those of the external display 100. By way of non-limiting example, such different or additional functions or purposes can include, but are not limited to, displaying at least some different (i.e., “secondary”) information in addition to or in lieu of some or all of the primary medical information displayed by the external display 100, wherein such secondary information can be entirely or partially medical in nature, or, instead, can be non-medical related.

This is shown in more detail in FIGS. 4A-4F, each of which depict an exemplary monitor device 200 in a docked mode (i.e., while docked within the docking station 300) wherein the monitor device 200 is configured to display various secondary information on its viewing screen 210 in response to predetermined user inputs. In FIG. 4A, the display screen 210 of the monitor device 200 depicts an exemplary “splash” image, which, by way of non-limiting example, can be displayed when the monitor device is initially powered up (e.g., in response to a user pressing the power button 230D). While the splash image of FIG. 4A is displayed, a user can input whether the patient being monitored is a new patient or not, such as by using the keypad buttons 230E-230H of the user interface in order to cause a cursor or icon (not shown) to selectively enter using either a “Yes” box 1010 or a “No” box 1020 and then pressing the enter button 230J while the appropriate choice is highlighted or otherwise selectable.

If a user indicates that this is not a new patient by choosing the “No” box 1020 at the FIG. 4A splash image, then the viewing/display screen 210 of the monitor device 200 will be caused to display some primary medical information and some secondary medical information in the form of waveforms 500 and/or numerics 510, such as those shown in FIG. 4B relating to the patient. In this example, several of the patient's vital signs are displayed. If a user selects (e.g., via the keypad buttons 230E-230H and/or the enter button 230J) the “main” icon 1030 depicted in FIG. 4B, then the screen 210 of the monitor device 200 will be caused to display main menu information, such as that which is shown in FIG. 4C.

The main menu information of FIG. 4C includes various icons 1040 each of which, if selected (e.g., via the keypad buttons 230E-230H and/or the enter button 230J), will cause the screen 210 of the monitor device 200 to display secondary information so as to enable a user to input data and/or to modify what is displayed by the monitor device.

For example, if the “Patient ADT” icon 1040A in FIG. 4C is selected (e.g., via the keypad buttons 230E-230H and/or the enter button 230J), then the screen 210 of the monitor device 200 will display certain patient information (e.g., name, age, gender, height, weight) and/or status information (e.g., code status, alarm status, etc.).

If a user selects (e.g., via the keypad buttons 230E-230H and/or the enter button 230J) the “Parameter Setup” icon 1040B in FIG. 4C, then the screen 210 of the monitor device 200 will appear as shown in FIG. 4D, which depicts a plurality of icons 1050 relating to medical parameters. If a user selects (e.g., via the keypad buttons 230E-230H and/or the enter button 230J) either the “Back” icon 1060 or the “Main” icon 1065 depicted in FIG. 4D, then the screen 210 of the monitor device 200 automatically will revert to its FIG. 4C configuration.

If a user selects (e.g., via the keypad buttons 230E-230H and/or the enter button 230J) the “Alarm Setup” icon 1040C of FIG. 4C, then a patient alarm can be enabled, disabled and/or muted.

If a user selects (e.g., via the keypad buttons 230E-230H and/or the enter button 230J) the “Procedures” icon 1040D of FIG. 4C, then the screen 210 of the monitor device 200 will display icons 1070 as shown in FIG. 4E so as to enable a user to select. (e.g., via the keypad buttons 230E-230H and/or the enter button 230J) one or more medical procedures to be performed at that time or to schedule one or more of such procedures to occur at a predetermined future time. If a user selects (e.g., via the keypad buttons 230E-230H and/or the enter button 230J) either the “Back” icon 1080 or the “Main” icon 1090 depicted in FIG. 4E, then the screen 210 of the monitor device 200 will revert to its FIG. 4C configuration.

If a user selects (e.g., via the keypad buttons 230E-230H and/or the enter button 230J) the “Standby” icon 1040E of FIG. 4C, then the monitor device 200 will enter standby mode in which the monitor device is temporarily paused (e.g., to allow a patient to visit the restroom or to eat a meal).

If a user selects (e.g., via the keypad buttons 230E-230H and/or the enter button 230J) the “Privacy” icon 1040F of FIG. 4C, then the screen 210 of the monitor device 200 will not display any information. This feature provides/protects patient privacy (e.g., if people are visiting the patient).

If a user selects (e.g., via the keypad buttons 230E-230H and/or the enter button 230J) the “Trend” icon 1040G of FIG. 4C, then various trends in previously measured information can be displayed on the screen 210 of the monitor device 200, can be downloaded, and/or can be printed.

If a user selects (e.g., via the keypad buttons 230E-230H and/or the enter button 230J) the “Display” icon 1040H of FIG. 4C, then what is displayed on the screen 210 of the monitor device 200 can be modified and or resized as desired.

If a user selects (e.g., via the keypad buttons 230E-230H and/or the enter button 230J) the “Settings” icon 1040J of FIG. 4C, then one or more settings (e.g., language, metric or non-metric, decimal or exponential) of the monitor device 200 can be modified, enabled or disabled.

If a user selects (e.g., via the keypad buttons 230E-230H and/or the enter button 230J) the “Print” icon 1040K of FIG. 4C, then various data can be caused to be printed and/or downloaded.

Lastly, if a user selects (e.g., via the keypad buttons 230E-230H and/or the enter button 230J) the “Exit” icon 1040L in FIG. 4C, then the screen 210 of the monitor device 200 will revert to its FIG. 4B configuration.

If, at the FIG. 4A screen, a user instead selects “yes” (e.g., via the keypad buttons 230E-230H and/or the enter button 230J) to indicate that this is a new patient, then the screen 210 of the monitor device 200 will display the self-explanatory icons 290 shown in FIG. 4F. It is understood, however, that the FIG. 4F screen 210 could be bypassed under certain exigent circumstances (e.g., is a patient is “crashing” or in distress) in order to enable more rapid monitoring of the patient.

The system 10 of FIG. 1 is configured such that the display screen 210 of the monitor device 200 depicts different information under different usage circumstances or conditions. By way of non-limiting example, and as is currently preferred, while the monitor device 200 is docked within the docking station 300 as shown in FIG. 1, the monitor device displays at least some secondary information (e.g., the information depicted in FIGS. 4A-4F) in addition to or in lieu of primary medical information, whereas when the monitor device is undocked or if power to the monitor device is discontinued, ceased or interrupted, the monitor device is caused (e.g., immediately and/or automatically) to display at least some primary medical information. Thus, through use of the monitoring system 10, one can beneficially obtain different, tailored medical information by selecting the mode of operation (i.e., portable or non-portable) of the monitor device 200.

There are various ways the monitor device 200 can be configured so as to enable its display screen 210 to display different information upon removing the monitor device 200 from the docking station 300 and/or upon discontinuation, cessation or interruption of power to the external display 100. For example, the monitor device 200 can be equipped with an A/C power cut-off such that once the monitor device senses a no A/C condition—as would occur if the monitor is dedocked or A/C power to the monitor is discontinued, ceased or interrupted—it automatically switches into a display mode in which it displays some, if not all of the primary medical information. Alternatively, the monitor device 200 can be equipped with a hardware pin shorted to ground while the monitor device is docked, and which, therefore, can trigger the monitor device to switch automatically into a display mode in which it displays some, if not all of the primary medical information upon sensing that ground is lost, as would occur if the monitor device is dedocked or if power to the monitor device was discontinued, ceased or interrupted. By way of another example, the monitor device 200 can be adapted to assess an expected voltage signal from the docking station 300, whereby if the signal falls out of an expected range—as would occur if the monitor is dedocked or A/C power to the monitor is discontinued, ceased or interrupted—the monitor device will be triggered to switch automatically into a display mode in which it displays some, if not all of the primary medical information upon sensing that ground is lost.

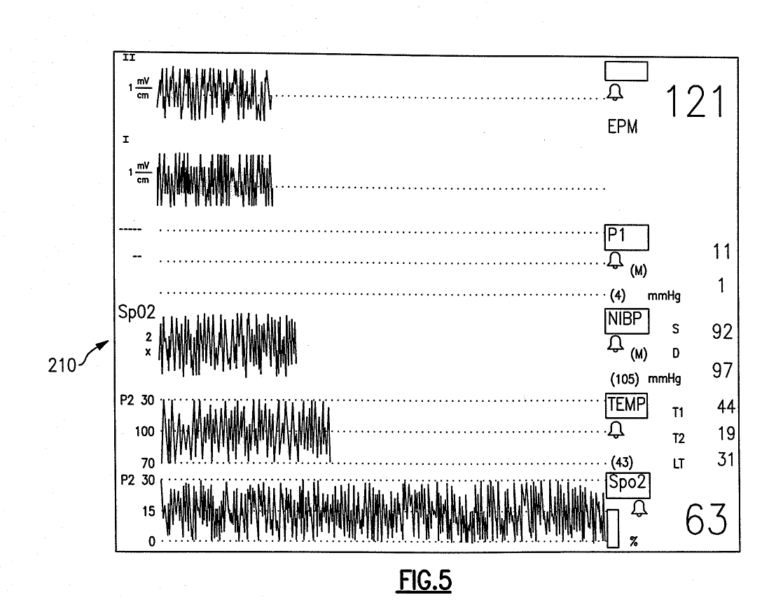

When one of these triggering conditions occurs, the screen 210 of the monitor device 200 automatically is caused or can be caused to display certain primary medical information. For example, FIG. 5 depicts the screen 210 of the monitor device 200 while in an undocked condition or under other circumstances (e.g., a power failure) such that the screen 210 has been caused to display substantially all of the primary medical information that is shown on the screen 110 of the external display 110 in FIG. 2. Although in FIG. 5 the screen 210 of the monitor device 200 depicts solely primary medical information, it is understood that the screen instead could be adapted to depict secondary information in addition to the primary medical information, or in lieu of certain primary medical information (e.g., if it is deemed unnecessary to monitor one or more of the “vital signs” of a patient).

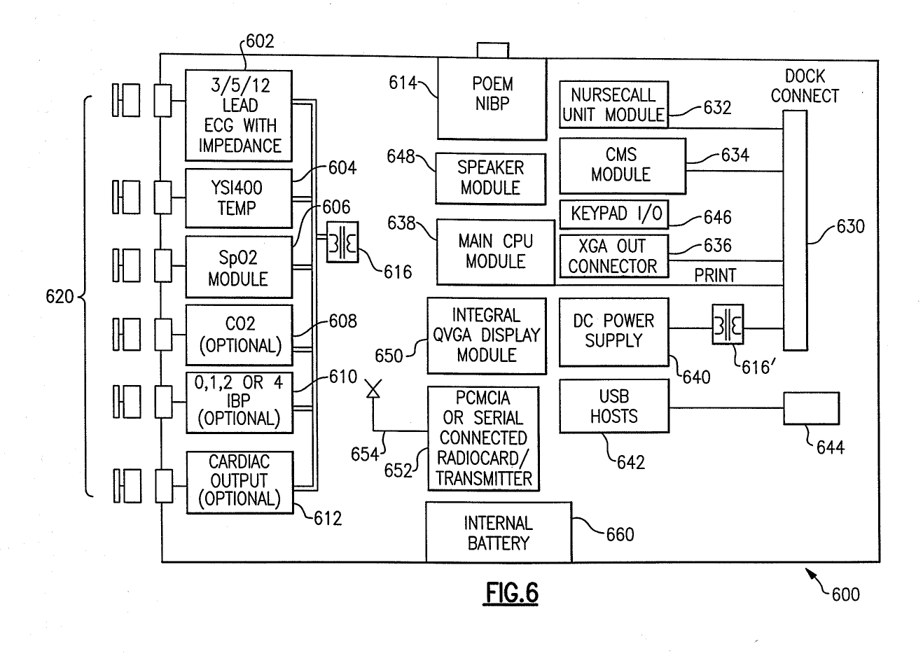

FIG. 6 is a drawing showing an exemplary block diagram 600 of the monitor device 200. In accordance with the exemplary embodiment of FIG. 6, there are included a plurality of inputs, including: (i) a 3/5/12 lead ECG with Impedance RESP module 602, (ii) a YSI TEMP module 604, (iii) an SpO₂ module 606, (iv) an LC101 CO₂ module 608; (v) 0, 1, 2, 4 or more invasive blood pressure (IBP) modules 610; (vi) a Cardiac Output module 612, and/or (vii) a POEM NIBP module 614.

Each of these exemplary modules 602-614 corresponds to a segment of primary medical information and is configured to receive one or more signals for or representative of such information. For example, module 602 is configured to receive signals useful to measure electrocardiograms using any of 3, 5 or 12 input leads. Module 604 is configured to receive signals useful to measure at least one body temperature of a patient. Module 606 is configured to receive signals useful to measure SpO₂ (pulse oximetry) using, for example, a Masimo unit or a Nellcor unit, each of which is well known in the art. Module 608 is configured to receive signals useful to measure end-tidal CO₂ in a patient. Module 610 is configured to receive signals useful to measure blood pressure in a patient using one or more invasive procedures. Module 612 is configured to receive signals useful to measure cardiac output in a patient, and module 614 is configured to receive signals useful to measure blood pressure using one or more noninvasive procedures.

As depicted and in accordance with an exemplary embodiment, each of the herein referred to exemplary modules 602-614 is provided with an electrical isolation module, which is represented by the inductor symbols 616, 616′. The electrical isolation modules 616, 616′ provide a way to reliably assure that no electrical signals that may be injurious or life threatening to a patient will appear at any point on an instrument, or on any lead connecting a person to an instrument. It is well known in the art to have such electrical isolation, e.g., to comply with applicable FDA requirements for medical instruments and devices. A plurality of connectors 620 also are depicted in FIG. 6 and function to externally connect various cables or wires (not shown) to the monitor device 200, such as cables or wires to or from medical instrumentation. These connectors 620 generally are identical in number, purpose and location to those represented in FIG. 4 with reference numeral 220.

The components on the depicted dock connect 630 layout include devices, programs, and signals internal to the monitordevice 200 that are useful for communications to and/or from the monitor device. The dock connect 630 provides a path to pass signals through the docking station 300 to their source sites external to the apparatus, and, possibly, external to the system 10.

The NurseCall unit 632 is a module useful to communicate according to a NurseCall protocol. The NurseCall protocol is a connectivity solution commonly used in Europe and Asia that provides simple alarm notification to the hospital through an installed facility NurseCall interface/system. Internally to the device it is a simple OPEN/CLOSED communication portal where alarms from the device open the communication to send a single that an alarm is occurring. At the facility, this will trigger a light and/or sound outside the patient location that alerts the staff of a patient who has an alarming condition.

CMS (central monitoring station) module 634 is a central station connectivity solution defined by or unique to the developer. By way of non-limiting example, and as currently preferred, the CMS module 634 comprises connectivity solutions as used by Acuity central monitoring stations manufactured by Welch Allyn, Inc. of Beaverton, Oreg., whereby patient data, situations and alarms are available remotely to medical staff through a dedicated computer system (not shown). The monitor device 200 can be hardwire-connected to provide this connectivity through, e.g., an RS422 or a USB connection (see, e.g., connection 220J in FIG. 4) that connects to the computer system. In accordance with an alternative embodiment which does not require a signal to pass through the dock connect 630, there can be a wireless interface between the device and the computer system, e.g., through an integrated PCMCIA or Serial connected radio card/transmitter 652 and an antenna 654.

An isolated DC Power supply 640 is internal to the monitor device 200 and connects to an A/C outlet by way of electrical isolation module 616′ (e.g., via a power pass-through located in the docking station 300). This is useful in powering the monitor device 200 while it is docked in the docking station 300 and in recharging the monitor device's internal battery 660 for use during power outages and/or while the monitor device is used portably (i.e., while undocked from the docking station 300).

The XGA OUT 636 is a connector useful to make a connection between an external XGA device and the monitor device 200. Signals are passed through the docking station 300 to the connection to the external display 100. It is through this connection that the external display 100 receives the output from the device, including the screen layout, the menu decisions, and patient waveforms and vital signs data for display.

The MAIN CPU module 638 also operates through the docking station 300 by providing the output from the monitor device 200 to the docking station's printer and/or recorder in order to display, print and/or store patient vital signs, patient trends and/or ECG waveform data for recording, review and/or documentation purposes. The MAIN CPU module 638 also functions as the central processor that coordinates all the inputs and outputs of the total system 10 and integrates the data and power requirements for any of the parameter boards, including those in the monitor device 200.

In the exemplary embodiment depicted in FIG. 6, additional device components are found in the monitor device 200, including a SPEAKER module 648 and an INTEGRAL QVGA Display module 650. The SPEAKER module 648 is a patient safety component, required by law, to announce all alarming conditions with variations in sounds and tempo to alert the user to the level and type of alarm condition the device or patient that may be present at a given time. The INTEGRAL QVGA Display module 650, with optional touchscreen capabilities, is the internal device controller for the display screen 210 on the monitor device 200. When the patient monitor 200 is docked in the docking station 300, the INTEGRAL QVGA Display module 650 serves as the secondary screen, providing the method of user input and user interface with the system. When undocked or during periods of loss of power, the INTEGRAL QVGA Display module 650 converts to display on at least some, if not all, of the primary medical information, e.g., as shown in FIG. 5. Also, through the optional touchscreen or other keyboard/keypad/button 646 capability, the INTEGRAL QVGA Display module 650 allows access to the user interface as needed in the use of the monitor device 200.

USB HOSTs 642 and USB connectors 644 are connections placed within the patient monitor device 200 (or accessible on a surface of the device) for future upgrades and for other external device connections. KEYPAD I/O 646 is provided for signaling states of the monitor device's keys and buttons 230, which, as noted above, a user can employ to affect the system 10 as a whole, and/or its settings, connectivity connections, and external display through interactions with the device menus and settings.

A field-serviceable internal battery 660 provides the backup battery capabilities to maintain the monitor device 200 in an operational state while in portable mode (i.e., while undocked from the docking station 300) or during periods of loss of power or power interruption. It is currently preferred for the battery 660 to be a rechargeable battery, such as a Lithium ion battery, or a NiCad battery. In an emergency such as a power failure of some duration, a non-rechargeable battery may be substituted if operation is required for a period longer than the capacity of an available rechargeable battery.

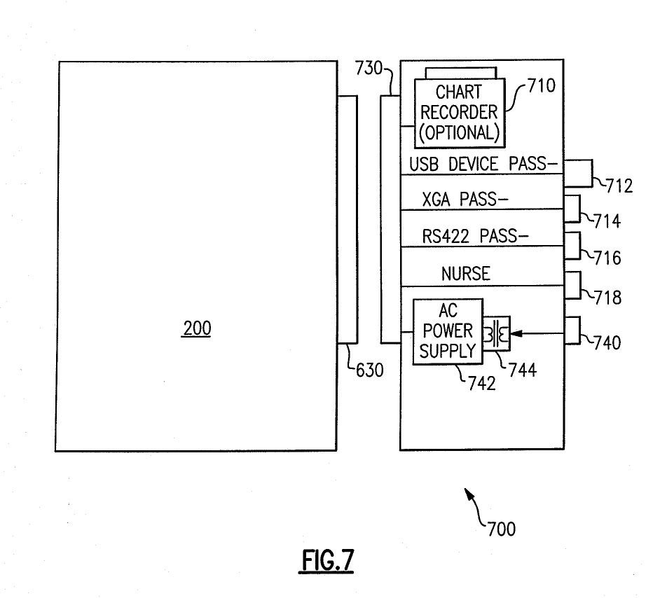

FIG. 7 is a drawing showing an exemplary docking station architecture in the form of block diagram 700. The docking station architecture provides connections for signals to pass between the dock connect module 630 of the monitor device 200 and the docking station 300.

In FIG. 7, the monitor device 200 is shown having a dock connect 630 that mates with a connector 730 of the docking station 300. The dock connect 630 and the mating connector 730 can be any convenient mating connectors having a sufficient number of connecting contacts. By way of non-limiting example, one can use commercially available multi-pin connectors having sufficient numbers of contacts and contacts of sufficient contact size, area, shielding, and other features necessary to pass the required signals without degradation. Examples of such multi-pin connectors that are commonly used in the computer hardware arts include, but are not limited to USB connectors, 9-pin, 15-pin and 25 pin D connectors, multi-pin power supply connectors, and 64 pin connectors commonly used with data ribbon cable. The connector 730 may be keyed or otherwise designed to permit connection of the connector in only one orientation (e.g., to prevent damage to components by subjecting them by error to signals having incorrect magnitudes or polarities).

Data is sent from the MAIN CPU 638 of the monitor device 200 through the dock connect 630 to the docking station connector 730 to the integrated printer and/or optional chart recorder 710 inside the docking station 300. The other connections 730 are further pass through connectors from the dock connect 630 to the system 10. The pass through connectors 730 provide paths for, e.g., a USB passthrough 712, an XGA passthrough 714, an RS422 passthrough 716, and a NurseCall passthrough 718, each of which connects the monitor device 200 with these outside systems via the docking station 300 using the single connector 730, rather than having to detach numerous individual connections from the monitor device before the monitor device is used in a portable mode (i.e., while the monitor device is dedocked), such as when a patient is moved.

A power supply, such as AC power supply 742 can be provided, and can include an electrical isolation module 744. The AC power supply 742 and electrical isolation module 744 are connected to pins of the connector 730 that mate to the dock connect 630 to provide power to the DC power supply 640, and to receive power from a separate connector 740 that can be connected to a convenient source of power, such as a wall electrical socket providing conventional AC power (120 V, 60 Hertz in the U.S., other values elsewhere).

In operation, the MAIN CPU 638 directs the system 10 to identify the appropriate state of power/battery or system disconnect and to alert the displays to respond. In normal operation when the monitor device 200 is in a docked condition (i.e., is docked within the docking station 300) and is provided with AC/DC power, the external display 100 (through data provided through the XGA OUT connection) serves as the main, primary display, providing primary medical information and the monitor device 200 (INTEGRAL QVGA) will provide a user interface solution plus, if desired, secondary information (e.g., the information shown in FIGS. 4A-4F). In a condition when the monitor device 200 is either undocked or lacks power or an appropriate signal through dedocking, unplugging or power interruption, the MAIN CPU 638 identifies the condition, and causes the display screen 210 of the monitor device 200 (INTEGRAL QVGA) to automatically depict at least some primary medical information, such as that which is shown in FIG. 5. In such a condition, the docking station 300 and external display 100 remain dormant. When the system 10 is activated through redocking of the monitor device 200 or provision of appropriate input power, the MAIN CPU 638 identifies the restored conditions and configures the system 10 so that all components are active in their normal state.

Although various embodiments have been described herein, it is not intended that such embodiments be regarded as limiting the scope of the disclosure, except as and to the extent that they are included in the following claims—that is, the foregoing description is merely illustrative, and it should be understood that variations and modifications can be effected without departing from the scope or spirit of the various embodiments as set forth in the following claims. Moreover, any document(s) mentioned herein are incorporated by reference in its/their entirety, as are any other documents that are referenced within such document(s).

We claim:

1. A system, comprising:
a first display device;
a second display device; and
a docking station adapted to receive said second display device, wherein said second display device is operable in one of a docked mode while docked within said docking station and an undocked mode while undocked from said docking station, such that while in said undocked mode said second display device operates as a primary display device for said system, and such that while in said docked mode said second display device operates as a user interface and said first display device operates as said primary display device for said system.

2. The system of claim 1, wherein said first display device is a computer monitor.

3. The system of claim 1, wherein said second display device is a medical patient monitoring device.

4. The system of claim 1, wherein said second display device includes at least one power source for powering said second display device while said second display device is in said undocked mode.

5. The system of claim 4, wherein said at least one power source is at least one battery.

6. The system of claim 1, wherein said second display device further comprises:
a plurality of modules, wherein each of said plurality of modules is adapted to receive at least one signal; and
a microprocessor that includes software to permit each of said at least one signals to be displayed on said second display device in at least one display format.

7. The system of claim 1, further comprising:
a control unit adapted to sense when said second display device is in said undocked mode, said control unit including a communicating means for communicating display data to said first display device while said second display device is in said docked mode.

8. The system of claim 1, wherein said user interface is a touch screen.

9. The system of claim 1, wherein said user interface includes a plurality of user actuable controls, and wherein at least some of said user actuable controls enable a user to maneuver within and select from information displayed on said second display device.

10. The system of claim 9, wherein said plurality of user actuable controls includes at least one button selected from the group consisting of: (a) a power button, (b) a mute button, (c) an image save button, and a (d) perform procedure button.

11. The system of claim 1, wherein said docking station includes a receiving area to receive said second display device, said receiving area including at least one detent.

12. The system of claim 1, wherein said docking station includes a receiving slot.

13. The system of claim 1, wherein said docking station includes a dispensing slot.

14. A medical monitoring system, comprising:
a docking station having a receiving area; and
a monitor device adapted to fit within said receiving area of said docking station, wherein said monitor device includes a display screen on which medical information is displayed, and wherein said medical information is automatically modified upon removal of said monitor device from said docking station.

15. The medical monitoring system of claim 14, wherein said monitor device includes a plurality of receiving areas, and wherein each of said plurality of receiving areas enables connection between said monitor device and equipment adapted to measure at least one physiological parameter.

16. The medical monitoring system of claim 15, wherein said at least one physiological parameter is selected from the group consisting of: (a) pulse oximetry, (b) end-tidal CO₂, (c) heart rate, (d) non-invasive blood pressure, (e) invasive blood pressure, and (f) body temperature.

17. The medical monitoring system of claim 14, wherein said monitor device includes a user interface.

18. The medical monitoring system of claim 17, wherein said user interface is a touch screen.

19. The medical monitoring system of claim 17, wherein said user interface includes a plurality of user actuable controls, and wherein at least some of said plurality of user actuable controls enable a user to maneuver within and select from said medical information displayed on said monitor device.

20. The medical monitoring system of claim 19, wherein said plurality of user actuable controls includes at least one button selected from the group consisting of: (a) a power button, (b) a mute button, (c) an image save button, and a (d) perform procedure button.

21. The medical monitoring system of claim 14, wherein said receiving area of said docking station includes at least one detent.

22. The medical monitoring system of claim 14, wherein said docking station includes a receiving slot.

23. The medical monitoring system of claim 14, wherein said docking station includes a dispensing slot.

24. The medical monitoring system of claim 14, wherein said monitor device includes at least one power source for powering said monitor device upon removal of said monitor device from said docking station.

25. The medical monitoring system of claim 24, wherein said at least one power source is at least one battery.

26. The system of claim 14, wherein said monitor device further comprises:
a plurality of modules, wherein each of said plurality of modules is adapted to receive at least one signal; and
a microprocessor that includes software to permit each of said at least one signals to be displayed on said monitor device in at least one display format.

27. A medical monitoring system, comprising:
a docking station having a receiving area; and
a monitor device adapted to fit within said receiving area of said docking station, said monitor device comprising:
(a) a display screen;
(b) a plurality of receiving areas, wherein each of said plurality of receiving areas enables connection between said monitor device and equipment adapted to measure at least one physiological parameter;
(c) a plurality of modules, wherein each of said plurality of modules is adapted to receive at least one signal representative of medical information;
(d) a microprocessor including software to permit each of said at least one signal to be displayed on said monitor device in at least one display format; and
(e) a user interface including a plurality of user actuable controls,
wherein said medical information is automatically modified upon removal of said monitor device from the docking station.

28. A system comprising:
a first display device;
a second display device; and
a docking station adapted to receive said second display device, wherein said second display device is operable in one of a docked mode while docked within said docking station and an undocked mode while undocked from said docking station, such that while in said undocked mode said second display device operates as a primary display device for said system, and such that while in said docked mode said second display device operates any secondary display and said first display device operates as said primary display device for said system.

29. The system of claim 28, wherein said first display device is a computer monitor and said second display device is a patient monitoring device.

30. The system of claim 28, wherein said second display device includes at least one power source for powering said second display device when in said undocked mode.

31. The system of claim 28, wherein said second display device is configured to operate as a user interface when in said docked mode.

32. The system of claim 31, wherein said user interface is a touch screen.
